(19) United States
(12) Patent Application Publication
(10) Pub. No.: US 2021/0387070 A1
(43) Pub. Date: Dec. 16, 2021

(54) METHOD AND APPARATUS FOR BASEBALL STRATEGY PLANNING BASED ON REINFORCEMENT LEARNING

(71) Applicant: National Tsing Hua University, Hsinchu City (TW)

(72) Inventors: Wei-Yu Chiu, Hsinchu City (TW); Song-Jen Chen, Hsinchu City (TW)

(73) Assignee: National Tsing Hua University, Hsinchu City (TW)

(21) Appl. No.: 16/942,758

(22) Filed: Jul. 29, 2020

(30) Foreign Application Priority Data
Jun. 16, 2020 (TW) 109120133

(51) Int. Cl. A63B 71/06; G06F 16/22; G06F 16/21

(52) U.S. Cl. CPC A63B 71/0605; A63B 71/0669; A63B 2102/18; G06F 16/2282; G06F 16/219; A63B 71/0622

(57) ABSTRACT

A method and an apparatus for baseball strategy planning based on reinforcement learning are provided. The method includes steps below. Historical data of innings in past games of a team is collected. Multiple game states, multiple offensive and defensive actions, and multiple rewards corresponding to multiple offensive and defensive results are defined based on multiple offensive and defensive processes occurring during the game, and are used to establish a Q table. The Q table is updated according to multiple combinations of the game state, the offensive and defensive action, and the offensive and defensive result recorded in the historical data. According to a current game state, Q values of all offensive and defensive actions executable in the current game state recorded in the updated Q table are sorted, and the offensive and defensive action suitable for being executed in the current game state is recommended according to a sorting result.

### CROSS-REFERENCE TO RELATED APPLICATION

This application claims the priority benefit of Taiwan application serial no. 109120133, filed on Jun. 16, 2020. The entirety of the above-mentioned patent application is hereby incorporated by reference herein and made a part of this specification.

### BACKGROUND

### Technical Field

The disclosure relates to a reinforcement learning method and a reinforcement learning apparatus, and in particular, to a baseball strategy planning method and a baseball strategy planning apparatus based on reinforcement learning.

### Description of Related Art

In baseball games, multiple defense and offense strategies are available. Conventionally, the coaches decide the offensive and defensive strategies after weighing the pros and cons based on the current game situation and player qualities. However, it is difficult to assess in real-time whether the selected strategy contributes to a positive result, and it cannot be analyzed until the game is over.

At present, domestic and oversea researchers have proposed many techniques for evaluating baseball inning strategies by using learning methods, but most of the research is based on individuals (i.e., players) instead of the entire team as the learning object. For example, with respect to the performance of a player in the baseball game, after learning training, a strategy capable of improving the batting average can be obtained based on past experience to thereby help the team to win more scores. Although the strategies provided by these methods can improve individual game performance, they are not necessarily the optimal strategy for the team, considering that the entire game is constrained by various factors.

### SUMMARY

The disclosure provides a baseball strategy planning method and a baseball strategy planning apparatus based on reinforcement learning, which plan the overall offensive and defensive strategies of the team by using the reinforcement learning method and can evaluate and recommend the optimal strategy in the current game state in real-time.

The disclosure provides a baseball strategy planning method based on reinforcement learning adapted for an electronic apparatus having a processor. The method includes the following steps. Historical data of multiple innings in past games of a team is collected. Multiple game states, multiple offensive and defensive actions, and multiple rewards corresponding to multiple offensive and defensive results are defined according to multiple offensive and defensive processes occurring during the game, and are used to establish a Q table. The Q table is updated according to multiple combinations of the game state, the offensive and defensive action, and the offensive and defensive result recorded in the historical data. According to a current game state, Q values of all offensive and defensive actions executable in the game state recorded in the updated Q table are sorted, and the offensive and defensive action suitable for being executed in the game state is recommended according to a sorting result.

In an embodiment of the disclosure, the step of updating the Q table according to the multiple combinations of the game state, the offensive and defensive action, and the offensive and defensive result recorded in the historical data includes the following steps. For each of the game states, an offensive and defensive result and a new game state obtained after executing multiple offensive and defensive actions in the game state recorded in the historical data are searched for, and are used to calculate a reward obtained by executing each of the offensive and defensive actions in the game state. By using the calculated rewards and Q values of executing multiple offensive and defensive actions in the new game state, a Q value of executing each of the offensive and defensive actions in the game state in the Q table is updated.

In an embodiment of the disclosure, after the step of recommending the offensive and defensive action suitable for being executed in the game state according to the sorting result, the method further includes the following steps. A selection of the recommended offensive and defensive action is received. A reward obtained by executing the selected offensive and defensive action in the game state is calculated according to an offensive and defensive result and a new game state obtained after executing the selected offensive and defensive action. By using the calculated reward and Q values of executing multiple offensive and defensive actions in the new game state, a Q value of executing the selected offensive and defensive action in the game state in the Q table is updated.

The disclosure provides a baseball strategy planning apparatus based on reinforcement learning, including a data retrieval device, a storage device, and a processor. The data retrieval device is connected an external device. The storage device stores a computer program. The processor is coupled to the data retrieval device and the storage device and is configured to load and execute the computer program to perform the following steps. Historical data of multiple innings in past games of a team is collected by the data retrieval device from the external device. Multiple game states, multiple offensive and defensive actions, and multiple rewards corresponding to multiple offensive and defensive results are defined according to multiple offensive and defensive processes occurring during the game, and are used to establish a Q table. The Q table is updated according to multiple combinations of the game state, the offensive and defensive action, and the offensive and defensive result recorded in the historical data. According to a current game state, Q values of all offensive and defensive actions executable in the game state recorded in the updated Q table are sorted, and the offensive and defensive action suitable for being executed in the game state is recommended according to a sorting result.

In an embodiment of the disclosure, the game state includes a base occupation status, a number of outs, or a strike/ball count.

In an embodiment of the disclosure, the offensive and defensive action includes multiple pitch types of a pitcher and multiple hitting actions of a hitter, and the hitting actions include a bunt, a hit, a sacrifice fly, or no swing.

In an embodiment of the disclosure, the rewards corresponding to the offensive and defensive results include negative rewards representing losing a score, a base being advanced, and hitting by a hitter on a defensive side, a zero reward representing not losing a score on the defensive side, and positive rewards representing not being hit by the hitter, and striking out or putting out the hitter on the defensive side.

In an embodiment of the disclosure, the rewards corresponding to the offensive and defensive results include positive rewards representing scoring, advancing a base, and hitting a ball on an offensive side, a zero reward representing not scoring on the offensive side, and negative rewards representing a hitter missing a ball, and being stricken out or put out on the offensive side.

In an embodiment of the disclosure, the Q values of all offensive and defensive actions executable in the game state include Q values of executing the offensive and defensive actions by multiple players capable of executing the offensive and defensive actions.

To make the aforementioned more comprehensible, several embodiments accompanied with drawings are described in detail as follows.

### BRIEF DESCRIPTION OF THE DRAWINGS

FIG. 1 is a block diagram showing a baseball strategy planning apparatus based on reinforcement learning according to an embodiment of the disclosure.

FIG. 2 is a flowchart showing a baseball strategy planning method based on reinforcement learning according to an embodiment of the disclosure.

FIG. 3 is a flowchart showing a method of updating the Q table according to an embodiment of the disclosure.

FIG. 4 is a flowchart showing an online learning method according to an embodiment of the disclosure.

### DESCRIPTION OF THE EMBODIMENTS

An embodiment of the disclosure provides a baseball strategy planning method and a baseball strategy planning apparatus based on reinforcement learning (RL), which use a reinforcement learning algorithm to generate offensive and defensive strategies in real-time in baseball innings. The method is divided into two stages. The first stage is offline planning, which collects past game data of the team, and updates a value function pairing the state and the action in the inning through reinforcement learning. The second stage is online learning, which uses the value function established in the first stage to recommend an optimal offensive or defensive strategy in the current state, and then further updates the value function pairing the state and the action in the inning according to the action actually selected.

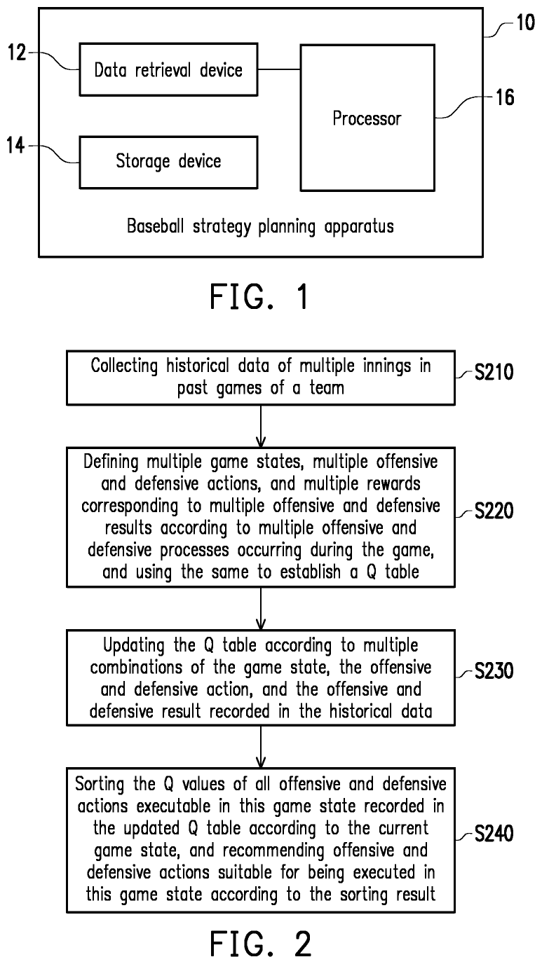

Specifically, FIG. 1 is a block diagram showing a baseball strategy planning apparatus based on reinforcement learning according to an embodiment of the disclosure. Referring to FIG. 1, a baseball strategy planning apparatus 10 according to the embodiment of the disclosure is, for example, a computing apparatus having computational capacities, such as a file server, a database server, an application server, a workstation, or a personal computer, and the baseball strategy planning apparatus 10 includes a data retrieval device 12, a storage device 14, a processor 16, etc. The functions of these components are described as follows.

The data retrieval device 12 is, for example, any wired or wireless interface device that may be connected to an external device (not shown) and is configured to collect historical data of multiple innings in past games of the team. In the case of a wired interface, the data retrieval device 12 may be an interface such as a universal serial bus (USB), RS232, a universal asynchronous receiver/transmitter (UART), an inter-integrated circuit (I2C), a serial peripheral interface (SPI), a display port, a thunderbolt, etc., but is not limited thereto. In the case of a wireless interface, the data retrieval device 12 may be device compatible with communication protocols such as wireless fidelity (Wi-Fi), RFID, bluetooth, infrared, near-field communication (NFC), device-to-device (D2D), etc., but is not limited thereto. In some embodiments, the data retrieval device 12 may also include a network card compatible with Ethernet or wireless network standards such as 802.11g, 802.11n, 802.11ac, etc., so that the baseball strategy planning apparatus 10 can be connected to an external device via a network to collect or receive historical information of baseball games.

The storage device 14 is, for example, any form of a fixed or movable random access memory (RAM), read-only memory (ROM), flash memory, hard disk, a similar device, or a combination of the above devices, and is configured to store a computer program executable by the processor 16. In some embodiments, the storage device 14 also stores, for example, historical information of baseball games collected by the data retrieval device 12 from an external device.

The processor 16 is, for example, a central processing unit (CPU), or another programmable general-purpose or specific-purpose microprocessor, microcontroller, digital signal processor (DSP), programmable controller, application specific integrated circuit (ASIC), programmable logic device (PLD), another similar device, or a combination of the above devices, and the disclosure is not limited thereto. In this embodiment, the processor 16 may load a computer program from the storage device 14 to execute the baseball strategy planning method based on reinforcement learning of the embodiment of the disclosure.

FIG. 2 is a flowchart showing a baseball strategy planning method based on reinforcement learning according to an embodiment of the disclosure. Referring to FIG. 1 and FIG. 2 at the same time, the method of this embodiment is applicable to the above baseball strategy planning apparatus 10, and the steps of the baseball strategy planning method of this embodiment will be described in detail below with reference to the components of the baseball strategy planning apparatus 10.

In step S210, the processor 16 of the baseball strategy planning apparatus 10 collects, through the data retrieval device 12, historical data of multiple innings in past games of a team from an external device. The external device is, for example, a server or a computer which records game data of each team and is not specifically limited herein.

In step S220, according to multiple offensive and defensive processes occurring during the game, the processor 16 defines multiple game states, multiple offensive and defensive actions, and multiple rewards corresponding to multiple offensive and defensive results, which are used to establish a Q table. Specifically, for example, in the embodiment of the disclosure, the game process is regarded as a Markov decision process (MDP), in which the time interval is defined as the pitching interval of the pitcher, and an episodic setting is adopted to define multiple combinations of a state, an action, and a reward respectively for the defensive and offensive processes, which are recorded in a Q table for learning.

Taking the Q table in Table 1 as an example, when the team takes an action A₀ in a state S₀, the team may obtain a reward R₁ according to the result and enter a next state S₁. Similarly, when the team takes an action A₁ in the state S₁, the team may obtain a reward R₂ according to the result and enter a next state S₂; when the team takes an action A₂ in the state S₂, the team may obtain a reward R₃ according to the result and enter a next state S₃, and so on. Therefore, a Q table which records the rewards obtained by taking various actions in various states can be established.

TABLE 1

| State | Action | Reward |
|---|---|---|
| S₀ | A₀ | R₁ |
| S₁ | A₁ | R₂ |
| S₂ | A₂ | R₃ |
| S₃ | A₃ | R₄ |
| . | . | . |
| . | . | . |
| . | . | . |
| . | . | . |

In some embodiments, the game state includes a base occupation status, a number of outs, a strike/ball count, or other information facilitating analysis of the situation, which is not specifically limited herein. The base occupation status includes, for example, no one on base and eight permutations/combinations of first base occupied, second base occupied, and third base occupied (i.e., nine possibilities in total), which are respectively defined as values of 0 to 8. The number of outs comes in, for example, three possibilities including zero outs, one out, and two outs, which are respectively defined as values of 0 to 2. The strike/ball count comes in, for example, twelve possibilities including the number of strikes (0 to 2) and the number of balls (0 to 3), which are respectively defined as values of 0 to 11. In an embodiment, the game state may record the above combination in a vector form. For example, when a player is on the first base, two players are out, and the count is two strikes and three balls, the game state may be recorded as {1, 2, 11}, and so on. In an embodiment, the game state is represented by, for example, one single value calculated from the above value combination, which is not specifically limited herein.

In some embodiments, the offensive and defensive actions may be divided depending on the defensive side and the offensive side. For the defensive side, the offensive and defensive actions include multiple pitch types of the pitcher, such as a straight pitch, a curveball, a slider, a forkball, etc. For the offensive side, the offensive and defensive actions include multiple hitting actions of the hitter, such as a bunt, a hit, a sacrifice fly, no swing, etc. The above offensive and defensive actions may be represented by different values. This embodiment does not limit the types of the offensive and defensive actions and their representation methods.

In some embodiments, the offensive and defensive results may also be divided depending on the defensive side and the offensive side, and according to situations favorable for the defensive side or the offensive side, negative-to-positive ranging rewards (including a zero reward) may be respectively given in this embodiment. A positive reward means that the situation is more favorable for the defensive side or the offensive side, a negative reward means that the situation is less favorable for the defensive side or the offensive side, and a zero reward means that the situation is neither favorable nor unfavorable for the defensive side or the offensive side.

For the defensive side, the rewards corresponding to the offensive and defensive results include negative rewards representing losing a score, a base being advanced, and hitting by the hitter, a zero reward representing not losing a score, and positive rewards representing not being hit by the hitter, and striking out or putting out a hitter. For example, whenever one score is lost, a reward β₁ is given; whenever one base is advanced by the opponent (including a base stolen by the runner), a reward β₂ is given; if the pitcher's ball is hit by the hitter, a reward β₃ is given; if a score is not lost, a reward 0 is given; if the pitcher's ball is not hit by the hitter, a reward β₄ is given; if the hitter is stricken out or put out, a reward β₅ is given, where β₁≤β₂≤β₃≤0≤β₄≤β₅.

On the other hand, for the offensive side, the rewards corresponding to the offensive and defensive results include positive rewards representing scoring, advancing a base, and hitting a ball, a zero reward representing not scoring, and negative rewards representing the hitter missing a ball, and being stricken out or put out. For example, if the hitter is stricken out or put out, a reward δ₁ is given; if the hitter swings but misses the ball, a reward δ₂ is given; if our side does not score, a reward 0 is given; if the hitter swings and hits the ball, a reward δ₃ is given; whenever our side advances one base (including a base stolen by the runner), a reward δ₄ is given; whenever our side scores one point, a reward δ₅ is given, where δ₁≤δ₂≤0≤δ₃≤δ₄≤δ₅.

Returning to the flowchart of FIG. 2, in step S230, according to the game states, the offensive and defensive actions, and the rewards corresponding to the offensive and defensive results as defined above, the processor 16 may update the Q table according to multiple combinations of the game state, the offensive and defensive action, and the offensive and defensive result recorded in the historical data. Specifically, for example, for each game state, the processor 16 searches for an offensive and defensive result and a new game state obtained after executing multiple offensive and defensive actions in the game state as recorded in the historical data, and uses them to calculate a reward obtained by executing each offensive and defensive action in the game state. Then, by using the calculated rewards and the Q values of executing multiple offensive and defensive actions in the new game state, the processor 16 updates the Q value of executing each offensive and defensive action in the game state in the Q table.

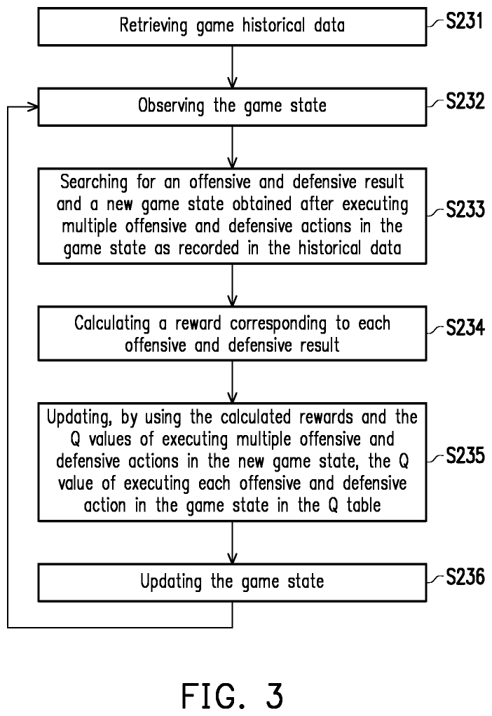

FIG. 3 is a flowchart showing a method of updating the Q table according to an embodiment of the disclosure. Referring to FIG. 2 and FIG. 3 at the same time, this embodiment describes detailed steps of step S230 in FIG. 2 above.

In step S231, the processor 16 accesses the storage device 12 to retrieve game historical data previously collected and stored in the storage device 12.

In step S232, the processor 16 observes the game state. The processor 16, for example, selects a game state for learning from multiple game states recorded in a previously established Q table.

In step S233, the processor 16 searches for an offensive and defensive result and a new game state obtained after executing different offensive and defensive actions in the game state as recorded in the historical data. For example, in the state where no one is out and the bases are loaded, after the offensive side executes a bunt, a result of scoring one point and a new game state where one player is out and the second and third bases are occupied are obtained.

In step S234, the processor 16 calculates a reward corresponding to each offensive and defensive result. For example, for the defensive side, if the offensive and defensive result is losing one score, the obtained reward is β₁; if the offensive and defensive result is no score loss, the obtained reward is 0; if the offensive and defensive result is striking out the hitter, the obtained reward is β₅. In contrast, for the offensive side, if the offensive and defensive result is being stricken out, the obtained reward is δ₁; if the offensive and defensive result is no score, the obtained reward is 0; if the offensive and defensive result is scoring one point, the obtained reward is δ₅.

In step S235, by using the calculated rewards and the Q values of executing multiple offensive and defensive actions in the new game state, the processor 16 updates the Q value of executing each offensive and defensive action in the game state in the Q table.

In step S236, the processor 16 updates the game state. Namely, the previously observed or learned game state is updated to the new game state. Afterwards, returning to step S232, the processor 16 re-observes the game state and performs learning by using the historical data.

Specifically, for the defensive side, assuming that an action A_{t,defense} is executed in a game state S_{t,defense} in round t, the reward corresponding to the execution result is R_{t+1,defense}, and the corresponding new game state (i.e., the game state in round t+1) is S_{t+1,defense}, then the Q value Q_{defense}(S_{t,defense}, A_{t,defense}) corresponding to the state S_{t,defense} and the action A_{t,defense} in the Q table may be updated by the following formula (1):

$${Q}_{defense} ( {S}_{t , defense} , {A}_{t , defense} ) ← ( 1 - α ) \cdot {Q}_{defense} ( {S}_{t , defense} , {A}_{t , defense} ) + α \cdot \{ {R}_{t + 1 , defense} + γ \cdot {max}_{α} ( {S}_{t + 1 , defense} , a ) \} \qquad ( 1 )$$

In the formula, α is the learning rate, γ is the discount factor, Q_{defense}(S_{t+1,defense},a) is the Q value of executing an action a in the new game state S_{t+1,defense}. Among multiple actions a in the game state S_{t,defense}, by taking an action having the largest Q value as the optimal action a*, a reward obtained by executing the action a* in the new game state S_{t+1,defense} is fed back to the Q value corresponding to the action a* in the original game state S_{t,defense}. In addition, the learning rate α is any number having a value between 0 and 1 and determines the influence on the update of Q_{defense}(S_{t,defense}, A_{t,defense}), as illustrated in formula (1). The discount factor γ is, for example, any number having a value between 0.9 and 0.99 and may determine the ratio of the Q value of the new game state S_{t+1,defense} to the fed back reward.

On the other hand, for the offensive side, assuming that an action A_{t,offense} is executed in a game state S_{t,offense} in round t, the reward corresponding to the execution result is R_{t+1,offense}, and the corresponding new game state (i.e., the game state in round t+1) is S_{t+1,offense}, then the Q value Q_{offense}(S_{t,offense}, A_{t,offense}) corresponding to state S_{t,offense} and the action A_{t,offense} in the Q table may be updated by the following formula (2):

$${Q}_{offense} ( {S}_{t , offense} , {A}_{t , offense} ) ← ( 1 - α ) \cdot {Q}_{offense} ( {S}_{t , offense} , {A}_{t , offense} ) + α \cdot \{ {R}_{t + 1 , offense} + γ \cdot {max}_{α} {Q}_{offense} ( {S}_{t + 1 , offense} , a ) \} \qquad ( 2 )$$

In the formula, α is the learning rate, γ is the discount factor, Q_{offense}(S_{t+1,offense},a) is the Q value of executing an action a in the new game state S_{t+1,offense}. Among multiple actions a in the game state S_{t,offense}, by taking an action having the largest Q value as the optimal action a*, a reward obtained by executing the action a* in the new game state S_{t+1,offense} is fed back to the Q value corresponding to the action a* in the original game state S_{t,offense}. In addition, the learning rate α is any number having a value between 0 and 1 and determines the influence on the update of Q_{offense}(S_{t,offense},A_{t,offense}), as illustrated in formula (2). The discount factor γ is, for example, any number having a value between 0.9 and 0.99 and may determine the ratio of the Q value of the new game state S_{t+1,offense} to the fed back reward.

Based on the offline training of the above steps, the Q table has learned the value function (i.e., Q value) of executing various actions in various states. Therefore, in the actual game, by applying this Q table, it is possible to evaluate the current game state in real-time and recommend the optimal strategy.

Specifically, returning to the flowchart of FIG. 2, in step S240, according to the current game state, the processor 16 sorts the Q values of all offensive and defensive actions executable in this game state recorded in the updated Q table, and recommends offensive and defensive actions suitable for being executed in this game state according to the sorting result. In some embodiments, for example, the processor 16 may sort the offensive and defensive actions according to the Q values corresponding to the offensive and defensive actions, so as to display or prompt one or more offensive and defensive actions having higher Q values for the team to choose from.

Taking the defensive side as an example, for the current game state S_{t,defense}, all actions a executable in this game state may be queried from the Q table to sort the Q values Q_{defense}(S_{t,defense},a) of all actions a for strategy evaluation. The optimal defensive strategy action A_{t,defense}* may be defined as:

$${A}_{t , defense}^{*} = {argmax}_{a ∈ A ( {S}_{t , desfense} )} {Q}_{defense} ( {S}_{t , defense} , a ) \qquad ( 3 )$$

In some embodiments, due to the different pitch types which each pitcher is capable of, the set of actions a in the above formula may be changed according to the capability of the pitcher at the moment; namely, the pitcher's capability may be incorporated into the learning and decision-making. Similarly, for the offensive side, the set of all actions a executable in the current game state may also be changed according to the capability of the hitter at the moment; namely, the capability of the hitter may also be incorporated into the learning and decision-making.

Based on the above, from the team's standpoint, the method of this embodiment plans the overall offensive and defensive strategies of the team by using the reinforcement learning method. Different from the datafication method for individual players, the method of this embodiment is more comprehensive and favorable for keeping up with the game.

It is noted that in the actual game, in addition to applying the pre-learned Q table to evaluate the current game state in real-time and recommend the optimal strategy, the embodiment of the disclosure may further perform online learning and update on the trained Q table according to the strategy selected by the team to continuously learn the game experience.

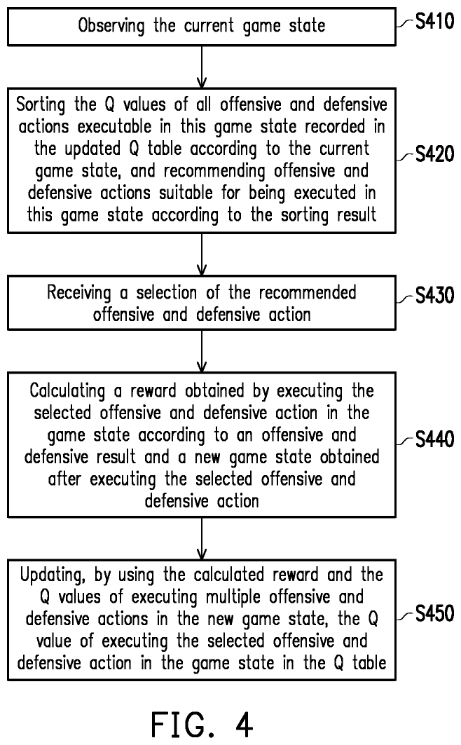

FIG. 4 is a flowchart showing an online learning method according to an embodiment of the disclosure. Referring to FIG. 2 and FIG. 4 at the same time, this embodiment describes the learning process after step S240 of FIG. 2 above.

In step S410, the processor 16 observes the current game state. The current game state is, for example, manually input by the coach, or obtained by the processor 16 through automatically reading information such as the inning score, the number of pitches, and the offensive and defensive data of the current game, which is not specifically limited herein.

In step S420, according to the current game state, the processor 16 sorts the Q values of all offensive and defensive actions executable in this game state recorded in the updated Q table, and recommends offensive and defensive actions suitable for being executed in this game state according to the sorting result. Step S420 is the same as or similar to step S240 in FIG. 2, so the details will not be repeatedly described herein.

Different from the foregoing embodiment, in this embodiment, in step S430, the processor 16 further receives a selection of the recommended offensive and defensive action. In some embodiments, the processor 16 receives the operation of selecting the recommended offensive and defensive action by the team (e.g., the coach) through an input device (not shown) such as a keyboard, a mouse, or a touch pad.

In step S440, the processor 16 calculates a reward obtained by executing the selected offensive and defensive action in the game state according to an offensive and defensive result and a new game state obtained after executing the selected offensive and defensive action. The processor 16 may also obtain the offensive and defensive result and the new game state through manually inputting or automatically reading information such as the inning score, the number of pitches, and the offensive and defensive data of the current game, which is not specifically limited herein.

In step S450, by using the calculated reward and the Q values of executing multiple offensive and defensive actions in the new game state, the processor 16 updates the Q value of executing the selected offensive and defensive action in the game state in the Q table.

Different from the offline planning stage which uses the actions selected in the past games to perform learning, in the online learning stage, the processor 16 directly calculates the reward to update the Q table according to the action currently selected by the team and the offensive and defensive result obtained after executing the action. By continuously updating the Q table, the Q table can continue to learn the game experience for evaluating or recommending strategies which meet the recent status of the team or the current status of the game in a future inning.

In summary of the above, in the baseball strategy planning method and the baseball strategy planning apparatus based on reinforcement learning in the embodiments of the disclosure, a Q table which can reflect pairing of states and actions in an inning is established in advance by using the past game data of the team, so that offensive or defensive strategies suitable for the current state can be recommended in an actual game. In addition, by continuously updating this Q table, it is possible to continue to learn the game experience and recommend strategies which are more in line with the current state of the game.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A baseball strategy planning method based on reinforcement learning, adapted for an electronic apparatus having a processor, the method comprising:
collecting historical data of multiple innings in past games of a team;
defining multiple game states, multiple offensive and defensive actions, and multiple rewards corresponding to multiple offensive and defensive results according to multiple offensive and defensive processes occurring during the game, and using the game states, the offensive and defensive actions, and the rewards to establish a Q table;
updating the Q table according to multiple combinations of the game state, the offensive and defensive action, and the offensive and defensive result recorded in the historical data; and
sorting, according to a current game state, Q values of all offensive and defensive actions executable in the game state recorded in the updated Q table, and recommending the offensive and defensive action suitable for being executed in the game state according to a sorting result.

2. The baseball strategy planning method based on reinforcement learning according to claim 1, wherein the game state comprises a base occupation status, a number of outs, or a strike/ball count.

3. The baseball strategy planning method based on reinforcement learning according to claim 1, wherein the offensive and defensive action comprises multiple pitch types of a pitcher and multiple hitting actions of a hitter, and the hitting actions comprise a bunt, a hit, a sacrifice fly, or no swing.

4. The baseball strategy planning method based on reinforcement learning according to claim 1, wherein the rewards corresponding to the offensive and defensive results comprise negative rewards representing losing a score, a base being advanced, and hitting by a hitter on a defensive side, a zero reward representing not losing a score on the defensive side, and positive rewards representing not being hit by the hitter, and striking out or putting out the hitter on the defensive side.

5. The baseball strategy planning method based on reinforcement learning according to claim 1, wherein the rewards corresponding to the offensive and defensive results comprise positive rewards representing scoring, advancing a base, and hitting a ball on an offensive side, a zero reward representing not scoring on the offensive side, and negative rewards representing a hitter missing a ball, and being stricken out or put out on the offensive side.

6. The baseball strategy planning method based on reinforcement learning according to claim 1, wherein the step of updating the Q table according to the multiple combinations of the game state, the offensive and defensive action, and the offensive and defensive result recorded in the historical data comprises:
for each of the game states, searching for an offensive and defensive result and a new game state obtained after executing multiple offensive and defensive actions in the game state recorded in the historical data, and using the offensive and defensive result and the new game state to calculate a reward obtained by executing each of the offensive and defensive actions in the game state; and
updating, by using the calculated rewards and Q values of executing multiple offensive and defensive actions in the new game state, a Q value of executing each of the offensive and defensive actions in the game state in the Q table.

7. The baseball strategy planning method based on reinforcement learning according to claim 1, wherein after the step of recommending the offensive and defensive action suitable for being executed in the game state according to the sorting result, the method further comprises:
receiving a selection of the recommended offensive and defensive action;
calculating a reward obtained by executing the selected offensive and defensive action in the game state according to an offensive and defensive result and a new game state obtained after executing the selected offensive and defensive action; and
updating, by using the calculated reward and Q values of executing multiple offensive and defensive actions in the new game state, a Q value of executing the selected offensive and defensive action in the game state in the Q table.

8. The baseball strategy planning method based on reinforcement learning according to claim 1, wherein the Q values of all offensive and defensive actions executable in the game state comprise Q values of executing the offensive and defensive actions by multiple players capable of executing the offensive and defensive actions.

9. A baseball strategy planning apparatus based on reinforcement learning, comprising:
a data retrieval device connected an external device;
a storage device storing a computer program; and
a processor coupled to the data retrieval device and the storage device and configured to load and execute the computer program to:
collect, by the data retrieval device, historical data of multiple innings in past games of a team from the external device;
define multiple game states, multiple offensive and defensive actions, and multiple rewards corresponding to multiple offensive and defensive results according to multiple offensive and defensive processes occurring during the game, and using the game states, the offensive and defensive actions, and the rewards to establish a Q table;
update the Q table according to multiple combinations of the game state, the offensive and defensive action, and the offensive and defensive result recorded in the historical data; and
sort, according to a current game state, Q values of all offensive and defensive actions executable in the game state recorded in the updated Q table, and recommend the offensive and defensive action suitable for being executed in the game state according to a sorting result.

10. The baseball strategy planning apparatus based on reinforcement learning according to claim 9, wherein the game state comprises a base occupation status, a number of outs, or a strike/ball count.

11. The baseball strategy planning apparatus based on reinforcement learning according to claim 9, wherein the offensive and defensive action comprises multiple pitch types of a pitcher and multiple hitting actions of a hitter, and the hitting actions comprise a bunt, a hit, a sacrifice fly, or no swing.

12. The baseball strategy planning apparatus based on reinforcement learning according to claim 9, wherein the rewards corresponding to the offensive and defensive results comprise negative rewards representing losing a score, a base being advanced, and hitting by a hitter on a defensive side, a zero reward representing not losing a score on the defensive side, and positive rewards representing not being hit by the hitter, and striking out or putting out the hitter on the defensive side.

13. The baseball strategy planning apparatus based on reinforcement learning according to claim 9, wherein the rewards corresponding to the offensive and defensive results comprise positive rewards representing scoring, advancing a base, and hitting a ball on an offensive side, a zero reward representing not scoring on the offensive side, and negative rewards representing a hitter missing a ball, and being stricken out or put out on the offensive side.

14. The baseball strategy planning apparatus based on reinforcement learning according to claim 9, wherein the processor is configured to:
for each of the game states, search for an offensive and defensive result and a new game state obtained after executing multiple offensive and defensive actions in the game state recorded in the historical data, and use the offensive and defensive result and the new game state to calculate a reward obtained by executing each of the offensive and defensive actions in the game state; and
update, by using the calculated rewards and Q values of executing multiple offensive and defensive actions in the new game state, a Q value of executing each of the offensive and defensive actions in the game state in the Q table.

15. The baseball strategy planning apparatus based on reinforcement learning according to claim 9, wherein the processor is further configured to:
receive a selection of the recommended offensive and defensive action;
calculate a reward obtained by executing the selected offensive and defensive action in the game state according to an offensive and defensive result and a new game state obtained after executing the selected offensive and defensive action; and
update, by using the calculated reward and Q values of executing multiple offensive and defensive actions in the new game state, a Q value of executing the selected offensive and defensive action in the game state in the Q table.

16. The baseball strategy planning apparatus based on reinforcement learning according to claim 9, wherein the Q values of all offensive and defensive actions executable in the game state comprise Q values of executing the offensive and defensive actions by multiple players capable of executing the offensive and defensive actions.
